(19) United States
(12) Patent Application Publication
(10) Pub. No.: US 2011/0184027 A1
(43) Pub. Date: Jul. 28, 2011

(54) TISSUE SELECTIVE STEAROYL-COA DESATURASE 1 INHIBITORS AND CELL BASED SCREENING ASSAY FOR THEIR IDENTIFICATION

(75) Inventors: Neelima Khairatkar-Joshi, Thard, IN (US); Daisy Shah, Dadar (IN); Laxmikant Atmaram Gharat, Thane (IN); Lakshminarayana Narayana, Navi Mumbai (IN)

(73) Assignee: GLENMARK PHARMACEUTICALS S.A., LA CHAUX-DE FONDS (CH)

(21) Appl. No.: 13/120,464

(22) PCT Filed: Sep. 25, 2008
(86) PCT No.: PCT/IB08/02510

§ 371 Date: Mar. 23, 2011

(51) Int. Cl. A61K 31/4439; C12N 5/071; C12Q 1/02; A61K 31/426; A61P 3/06; A61P 3/04; A61P 3/00; A61P 3/08; A61P 9/00; A61P 9/10; A61P 9/12; A61P 3/10

(52) U.S. Cl. 514/342; 435/375; 435/29; 514/365

(57) ABSTRACT

The present invention provides a method of treating a disorder treatable by administering a therapeutically effective amount of a tissue selective Stearoyl-CoA Desaturase 1 (SCD-1) modulator to a subject. In one embodiment, the present invention provides a method of treating a disorder treatable by administering a pharmaceutically effective amount of a tissue selective SCD-1 inhibitor to a subject. The invention also provides whole cell based screening assays to identify agents that selectively modulate the activity of SCD-1. This invention further provides whole cell based screening assays to identify agents that differentially inhibit SCD in different body tissues.

### TECHNICAL FIELD

The present patent application provides a method for treating disorders or conditions with tissue selective Stearoyl-CoA Desaturase 1 (SCD-1) modulators. This patent application further provides whole cell based screening assays to identify agents that differentially inhibit SCD-1 in targeted body tissues.

### BACKGROUND

Primary defects in energy balance produce obesity which is manifested by increases in free fatty acids and excessive lipid accumulation in some organs. Obesity is closely associated with increased risk for numerous conditions that shorten life, including diabetes, insulin resistance, coronary artery disease, hypertension and non-alcoholic fatty liver disease collectively known as metabolic syndrome (see, e.g., J. Am. Med. Assoc. 288, 1723-1727 (2002)).

Fatty acid desaturases convert saturated fatty acids to unsaturated fatty acids by introducing double bonds into growing fatty acid chains. SCD-1 in particular had been identified as a key rate-limiting enzyme that plays a role in lipid metabolism and body weight control (see, e.g., Science, 297 240-243 (2002); J Clinical Investigation, 1-9 (2005); Obesity Reviews, 6, 169-174 (2005)). SCD-1 is believed to catalyze biosynthesis of monounsaturated fatty acids from saturated fatty acid substrates by the addition of a cis double bond between carbons 9 and 10 of the fatty chain (see, e.g., Cum Drug Targets Immune Endocr Metabol Disord., 3, 271-280 (2003); PNAS, 71, 4565-4569 (1974). The preferred substrates, palmitoyl and stearoyl CoA, are converted to palmitoleoyl and oleoyl CoA, respectively. Oleate is believed to be the major monounsaturated fatty acid of membrane phospholipids, triglycerides, cholesterol esters, wax esters and alkyl-1,2-diacylglycerol.

The conversion of saturated fatty acids to unsaturated fatty acids is involved in lipogenesis, or burning of glucose to generate fatty acids. It is further believed the unsaturated fatty acids resulting from the SCD-1 action are then esterified with glycerol to produce triacylglycerols which are packaged in VLDL. Thus, SCD-1 is believed to be essential for the assembly of VLDL particles, which transport triacylglycerols from liver to adipose tissue and other sites. Based on the foregoing, a decrease in SCD-1 activity is believed to activate metabolic pathways that promote fatty acid β-oxidation and decrease lipogenesis in liver.

Several cell-free and cell based assays for studying SCD-1 modulators are reported in literature (see, e.g., Analytical Biochem, 29, 300-304 (1969); J. Biol. Chem., 276, 39455-39461 (2001); PNAS, 100, 11110-15 (2003); US Publication No. 2006/0281071; U.S. Pat. No. 6,759,208; U.S. Pat. No. 6,987,001; Nutrient Gene Expression, 1920-1924 (2000). Direct demonstration of SCD-1 inhibition by showing the absence of oleic acid product formation from ³H or ¹⁴C-stearic acid challenged cells has been attempted by some investigators (Nutrient Gene Expression, 1920-1924 (2000); Journal of Lipid Research 45, 972-980 (2004)). However, most of them involve laborious and complex organic solvent extractions to separate the unconsumed fatty acid substrate from the product. Also they are non-biological methods based on analytical techniques such as TLC/HPLC/GC. As a result, these assays may not be well adaptable for high through-put screening.

There exists a need for screening assay .that is more physiologically relevant than assays using cell free or recombinant SCD-1 enzyme preparation and that is useful for high through-put screening of SCD-1 modulators.

Also, there is a need for SCD-1 modulators with desired therapeutic profile.

### SUMMARY

In particular, the inventors of the present patent application have discovered a need for modulators of SCD-1 which do not induce undesired effects in tissues which are not targeted.

In a first aspect, there is provided a method of inhibiting lipid metabolism that proceeds via a Stearoyl-CoA Desaturase-1 (SCD-1) mediated pathway in a subject, the method including administering to the subject an inhibitory effective amount of a selective SCD-1 inhibitor that exhibits increased SCD-1 inhibitory activity in a target tissue in comparison with SCD-1 activity in a reference tissue.

In a second aspect, there is provided a method of decreasing serum levels of at least one lipid in a subject in need thereof, the method including administering to subject an inhibitory effective amount of a selective SCD-1 inhibitor that exhibits increased SCD-1 inhibitory activity in a target tissue in comparison with SCD-1 activity in a reference tissue. Preferably, the lipid of the second aspect is a triglyceride, LDL, HDL, VLDL or cholesterol.

Preferably, in the method according to the first and second aspects, the target tissue is liver, skin or cornea. Preferably, in the method according to the first and second aspects, the reference tissue is liver, skin or cornea. In one preferred variant, the target tissue is liver and the reference tissue is skin. In another preferred variant, the target tissue is liver and the reference tissue is cornea. While the invention is not limited by any specific theory, the selective SCD-1 inhibitor is believed to decrease conversion of saturated fatty acids to unsaturated fatty acids in liver cells to a greater extent than in skin cells. The preferred fatty acids are selected from palmitoyl CoA and stearoyl CoA.

In one variant in accordance with the first and the second aspects, the subject is a cell. In another variant, the subject is a mammal, preferably, a human.

In one embodiment of the first and the second aspects, the selective SCD-1 inhibitor is a small pharmaceutical molecule in the form of a free compound or pharmaceutically acceptable salt thereof. The non-limiting examples of SCD-1 inhibitors selective for liver include compounds of formula (I):

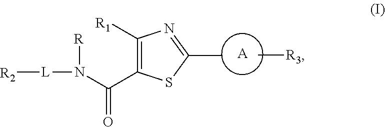

wherein A is a group selected from

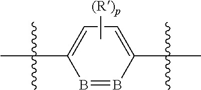

### or —C≡C—

at each occurrence B is independently selected from C or N, with proviso that at least one B is N;

R can be hydrogen, substituted or unsubstituted alkyl, substituted or unsubstituted cycloalkyl;

R′ can be is hydrogen, cyano, nitro, halogen, hydroxyl, substituted or unsubstituted alkyl, substituted or unsubstituted alkenyl, substituted or unsubstituted alkynyl;

‘p’ is an integer selected from 1-4;

R₁ can be hydrogen, substituted or unsubstituted alkyl, substituted or unsubstituted cycloalkyl, or haloalkyl;

R₂ can be substituted or unsubstituted aryl, substituted or unsubstituted heteroaryl substituted or unsubstituted cycloalkyl, substituted or unsubstituted heterocyclyl;

R₃ can be substituted or unsubstituted aryl, substituted or unsubstituted heteroaryl substituted or unsubstituted arylalkyl, substituted or unsubstituted heteroarylalkyl, substituted or unsubstituted cycloalkyl, substituted or unsubstituted heterocyclyl, substituted or unsubstituted cycloalkylalkyl, substituted or unsubstituted heterocyclylalkyl; and

L is alkylene linker which may be further substituted with halogen or alkyl. For SCD-1 inhibitors selective for liver, the preferred ratio of EC₃₅ for the SCD-1 inhibitor in skin cells to that in liver cells is greater than 1, more preferred ratio is at least 1.25:1, yet more preferred ratio is at least 2:1, yet more preferred ratio is at least 5:1.

While the invention is not limited to any specific theory, the SCD-1 inhibitor suitable for use in the methods of the first and second aspects may act through a variety of mechanisms of action. Thus, the SCD-1 inhibitor may be an inhibitor of SCD-1 gene expression, or an inhibitor of SCD-1 translation, or an inhibitor of SCD-1 enzyme, or a variant, or the SCD-1 inhibitor may exhibit selective permeability into the target tissue.

In a third aspect, there is provided a method of treating a disease or condition the treatment of which is effected or facilitated by inhibiting SCD-1 in a subject in need of the treatment, the method including administering to the subject a therapeutically effective amount of a selective SCD-1 inhibitor that exhibits increased SCD-1 inhibitory activity in a target tissue in comparison with SCD-1 activity in a reference tissue.

Preferably, in the method according to the third aspect, the target tissue is liver, skin or cornea. Preferably, in the method according to the third aspect, the reference tissue is liver, skin or cornea. In one preferred variant, the target tissue is liver and the reference tissue is skin. In another preferred variant, the target tissue is liver and the reference tissue is cornea. For SCD-1 inhibitors selective for liver, the preferred ratio of EC₃₅ for the SCD-1 inhibitor in skin cells to that in liver cells is greater than 1, more preferred ratio is at least 1.25:1, yet more preferred ratio is at least 2:1, yet more preferred ratio is at least 5:1. Preferably, the disorder or condition is obesity, diabetes, glucose tolerance; hyperinsulinemia; insulin insensitivity or resistance, metabolic syndromes, or cardiovascular disease. The preferred examples of cardiovascular disease are atherosclerosis, hypertension, lipidemia, dyslipidemia, elevated blood pressure, microalbuminemia, hyperuricaemia, hypercholesterolemia, hyperlipidemias, hypertriglyceridemias and arteriosclerosis.

In a forth aspect, there is provided a method of identifying a liver-selective SCD-1 inhibitor, the method including:
(a) assaying a test compound for SCD-1 inhibition in whole liver cells,
(b) assaying the test compound for SCD-1 inhibition in whole skin cells and/or in whole corneal cells, and
(c) selecting the test compound as a selective inhibitor of liver SCD-1 if the ratio of the EC₃₅ of the test compound in whole skin cells and/or corneal cells to that in whole liver cells is at least 1.2:1.
   Preferably, the compound is identified when the ratio is at least 2:1, more preferably, when the ratio is at least 3:1, yet more preferably, when the ratio is at least 4:1, yet more preferably, when the ratio is at least 5:1.

In a fifth aspect, there is provided a method of identifying a skin-selective SCD-1 inhibitor, the method including:

(a) assaying a test compound for SCD-1 inhibition in whole skin cells,

(b) assaying the test compound for SCD-1 inhibition in whole liver cells, and/or in whole corneal cells, and

(c) selecting the test compound as a selective inhibitor skin SCD-1 inhibitor if the ratio of the EC₃₅ in whole liver cells and/or corneal cells to that in whole skin cells is at least 1.2:1.

Preferably, the compound is identified when the ratio is at least 2:1, more preferably, when the ratio is at least 3:1, yet more preferably, when the ratio is at least 4:1, yet more preferably, when the ratio is at least 5:1.

In a sixth aspect, there is provided a method of identifying a corneal-selective SCD-1 inhibitor, the method including:
(a) assaying a test compound for SCD-1 inhibition in whole corneal cells,
(b) assaying the test compound for SCD-1 inhibition in whole liver cells, and/or in whole skin cells, and
(c) selecting the test compound as a selective corneal SCD-1 inhibitor if the ratio of the EC₃₅ in whole liver cells and/or skin cells to that in whole corneal cells is at least 1.2:1.
   Preferably, the compound is identified when the ratio is at least 2:1, more preferably, when the ratio is at least 3:1, yet more preferably, when the ratio is at least 4:1, yet more preferably, when the ratio is at least 5:1.

In a seventh aspect, there is provided a compound which is a selective SCD-1 inhibitor, wherein the selective SCD-1 inhibitor decreases conversion of saturated fatty acids to unsaturated fatty acids in liver cells to a greater extent than in skin cells and/or corneal cells, wherein the ratio of EC₃₅ for the SCD-1 inhibitor in skin cells and/or corneal cells to that in liver cells is greater than 1. For preferred inhibitors, the ratio is at least 1.25:1, more preferably, the ratio is at least 2:1, yet more preferably, the ratio is at least 5:1. The preferred liver-selective compounds have the structure:

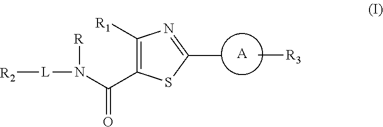

wherein,

A can be group selected from

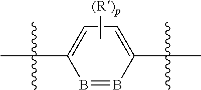

### or —C≡C—

at each occurrence B can be independently selected from C or N with proviso that atleast one B is N;

R can be hydrogen, substituted or unsubstituted alkyl, substituted or unsubstituted cycloalkyl;

R′ can be is hydrogen, cyano, nitro, halogen, hydroxyl, substituted or unsubstituted alkyl, substituted or unsubstituted alkenyl, substituted or unsubstituted alkynyl;

‘p’ is an integer selected from 1-4;

R₁ can be hydrogen, substituted or unsubstituted alkyl, substituted or unsubstituted cycloalkyl, or haloalkyl;

R₂ can be substituted or unsubstituted aryl, substituted or unsubstituted heteroaryl substituted or unsubstituted cycloalkyl, substituted or unsubstituted heterocyclyl;

R₃ can be substituted or unsubstituted aryl, substituted or unsubstituted heteroaryl substituted or unsubstituted arylalkyl, substituted or unsubstituted heteroarylalkyl, substituted or unsubstituted cycloalkyl, substituted or unsubstituted heterocyclyl, substituted or unsubstituted cycloalkylalkyl, substituted or unsubstituted heterocyclylalkyl;

L is alkylene linker which may be further substituted with halogen or alkyl.

In an eighth aspect, there is provided a pharmaceutical composition for treating a disease or condition the treatment of which is effected or facilitated by inhibiting SCD-1, the composition including i) a therapeutically effective amount of a selective SCD-1 inhibitor, wherein the selective SCD-1 inhibitor decreases conversion of saturated fatty acids to unsaturated fatty acids in liver cells to a greater extent than in skin cells and/or corneal cells, wherein the ratio of EC₃₅ for the SCD-1 inhibitor in skin cells and/or corneal cells to that in liver cells is greater than 1, and ii) a pharmaceutically acceptable carrier. Preferably, the pharmaceutical composition of this aspect contains a SCD-1 inhibitor which is a small pharmaceutical molecule in the form of a free compound or pharmaceutically acceptable salt thereof, more preferably, the small molecule is a compound of the formula (I):

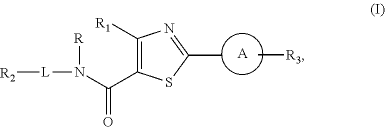

wherein A is

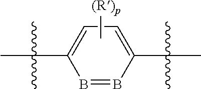

### or —C≡C—

at each occurrence B can be independently selected from C or N with proviso that atleast one B is N;

R can be hydrogen, substituted or unsubstituted alkyl, substituted or unsubstituted cycloalkyl;

R′ can be is hydrogen, cyano, nitro, halogen, hydroxyl, substituted or unsubstituted alkyl, substituted or unsubstituted alkenyl, substituted or unsubstituted alkynyl;

‘p’ is an integer selected from 1-4;

R₁ can be hydrogen, substituted or unsubstituted alkyl, substituted or unsubstituted cycloalkyl, or haloalkyl;

R₂ can be substituted or unsubstituted aryl, substituted or unsubstituted heteroaryl substituted or unsubstituted cycloalkyl, substituted or unsubstituted heterocyclyl;

R₃ can be substituted or unsubstituted aryl, substituted or unsubstituted heteroaryl substituted or unsubstituted arylalkyl, substituted or unsubstituted heteroarylalkyl, substituted or unsubstituted cycloalkyl, substituted or unsubstituted heterocyclyl, substituted or unsubstituted cycloalkylalkyl, substituted or unsubstituted heterocyclylalkyl;

L is alkylene linker which may be further substituted with halogen or alkyl.

### DETAILED DESCRIPTION

As used throughout this application, including the claims, the following terms have the meanings defined below, unless specifically indicated otherwise. The singular includes plural.

The term “inhibiting lipid metabolism that proceeds via an SCD-1-mediated pathway” denotes alteration in normal functioning of lipid metabolic pathway that involves SCD-1 enzyme, including, particularly, a decrease in lipid formation via a pathway facilitated by the SCD-1 enzyme.

The term “selective SCD-1 inhibitor” denotes a substance that affects activity of SCD-1 enzyme in a differential manner, particularly, a substance that selectively affects SCD activity in one tissue type as compared to SCD-1 activity of the same substance in another tissue type. For instance, a substance is a selective SCD-1 inhibitor if the SCD-1 inhibitory activity of the substance in target tissue is greater than SCD-1 inhibitory activity of the substance in reference tissue. An agent may be a selective inhibitor for liver SCD-1 by having a lower EC₃₅ in liver cells than in skin cells.

The term “target tissue” with respect to SCD-1 activity denotes a tissue type in which SCD-1 inhibition is intended to be exerted via administration of a substance having SCD-1 inhibitory activity. Non-limiting examples of “target tissue” include liver, skin and cornea.

The term “reference tissue” with respect to SCD-1 activity denotes a tissue type in which SCD-1 inhibition is not intended to be exerted via administration of a substance having SCD-1 inhibitory activity. Non-limiting example of a “reference tissue” include liver, skin and cornea.

The phrase “whole cells expressing SCD-1” includes cells which express the SCD-1 gene product endogenously. This includes cells which inherently contain an SCD-1 gene or have an SCD-1 gene.

The term EC₃₅ refers to the concentration of the compound which inhibits the activity of the enzyme half way between the baseline and maximum response of approximately 70%.

The term IC₅₀ refers to the concentration of inhibitor that reduces enzyme activity by 50%.

The term “pharmaceutically acceptable” means suitable for use in mammals.

The terms “patient”, “subject” and “mammal” are used interchangeably and refer to warm blooded animals such as, for example, guinea pigs, mice, rats, gerbils, cats, rabbits, dogs, monkeys, chimpanzees, stump tail macaques, and humans.

The term “treat” refers to the ability of the compounds to relieve, alleviate, ameliorate, or slow the symptoms or progression of the patient's disease (or condition) or any tissue damage associated with the disease.

The term “alkyl” refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl (isopropyl), n-butyl, n-pentyl, and 1,1-dimethylethyl (t-butyl). The term “C₁₋₆ alkyl” refers to an alkyl chain having 1 to 6 carbon atoms.

The term “alkenyl” refers to an aliphatic hydrocarbon group containing a carbon-carbon double bond and which may be a straight or branched chain having 2 to about 10 carbon atoms, e.g., ethenyl, 1-propenyl, 2-propenyl (allyl), iso-propenyl, 2-methyl-1-propenyl, 1-butenyl, and 2-butenyl.

The term “alkynyl” refers to a straight or branched chain hydrocarbyl radical having at least one carbon-carbon triple bond, and having 2 to about 12 carbon atoms (with radicals having 2 to about 10 carbon atoms being preferred), e.g., ethynyl, propynyl, and butynyl.

The term “haloalkyl” is used to denote a group comprised of an alkyl group substituted with halogen atom, where alkyl group is as defined above and halogen is used to denote fluorine, chlorine, bromine or iodine, an example of such group is trifluoromethyl, difluoromethyl.

The term “alkoxy” unless otherwise specified refers to an alkyl group attached via an oxygen linkage to the rest of the molecule. Representative examples of such groups are —OCH₃ and —OC₂H₅.

The term “haloalkoxy” unless otherwise specified refers to an haloalkyl group attached via an oxygen linkage to the rest of the molecule. Representative examples of such groups are —OCF₃ and —OC₂F₅.

The term “cycloalkyl” unless otherwise specified refers to substituted or unsubstituted non-aromatic mono or multicyclic ring system of 3 to about 20 carbon atoms, which may optionally contain one or more olefinic bonds unless constrained by the definition, such as cyclopropyl, cyclobutyl, cyclopentyl, and cyclohexyl. It also includes the cyclic ring system fused with an aryl ring, spiro systems. Examples of multicyclic cycloalkyl groups include, but are not limited to, perhydronapththyl, adamantyl and norbornyl groups, bridged cyclic groups and spirobicyclic groups, e.g., spiro (4,4) non-2-yl.

The term “cycloalkylalkyl” unless otherwise specified refers to substituted or unsubstituted cyclic ring-containing radical having 3 to about 20 carbon atoms directly attached to an alkyl group. The cycloalkylalkyl group may be attached to the main structure at any carbon atom in the alkyl group that results in the creation of a stable structure. Non-limiting examples of such groups include cyclopropylmethyl, cyclobutylethyl, and cyclopentylethyl.

The term “aryl” unless otherwise specified refers to substituted or unsubstituted carbocyclic aromatic radical having 6 to 14 carbon atoms, wherein the ring is mono-, bi-, or tricyclic, such as, but not limited to, phenyl, naphthyl, tetrahydronapthyl, indanyl, and biphenyl.

The term “arylalkyl” unless otherwise specified refers to substituted or unsubstituted aryl group as defined above directly bonded to an alkyl group as defined above, e.g., —CH₂C₆H₅ and —C₂H₅C₆H₅.

The term “heterocyclic ring” or “heterocyclyl” unless otherwise specified refers to substituted or unsubstituted non-aromatic 3 to 15 membered ring radical which consists of carbon atoms and from one to five heteroatoms selected from nitrogen, phosphorus, oxygen and sulfur. The heterocyclic ring radical may be a mono-, bi- or tricyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized; also, unless otherwise constrained by the definition the heterocyclic ring or heterocyclyl may optionally contain one or more olefinic bond(s). Examples of such heterocyclic ring radicals include, but are not limited to, azepinyl, azetidinyl, acridinyl, benzodioxolyl, benzodioxanyl, benzofurnyl, carbazolyl, cinnolinyl, dioxolanyl, indolizinyl, thienyl, naphthyridinyl, perhydroazepinyl, phenazinyl, phenothiazinyl, phenoxazinyl, indolyl, phthalazinyl, pyridyl, pteridinyl, purinyl, quinazolinyl, quinoxalinyl, quinolinyl, isoquinolinyl, tetrazoyl, imidazolyl, tetrahydroisquinolyl, piperidinyl, piperazinyl, 2-oxopiperazinyl, 2-oxopiperidinyl, 2-oxopyrrolidinyl, 2-oxoazepinyl, pyrrolyl, 4-piperidonyl, pyrrolidinyl, pyrazinyl, pyrimidinyl, pyridazinyl, oxazolyl, oxazolinyl, oxazolidinyl, triazolyl, indanyl, isoxazolyl, isoxazolidinyl, morpholinyl, thiazolyl, thiazolinyl, thiazolidinyl, isothiazolyl, quinuclidinyl, isothiazolidinyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzooxazolyl, furyl, tetrahydrofuryl, tetrahydropyranyl, benzothienyl, thiamorpholinyl, thiamorpholinyl sulfoxide, thiamorpholinyl sulfone, dioxaphospholanyl, oxadiazolyl, chromanyl, and isochromanyl. The heterocyclic ring radical may be attached to the main structure at any heteroatom or carbon atom that results in the creation of a stable structure.

The term “heteroaryl” unless otherwise specified refers to substituted or unsubstituted 5 to 14 membered aromatic heterocyclic ring radical with one or more heteroatom(s) independently selected from N, O or S. The heteroaryl may be a mono-, bi- or tricyclic ring system. The heteroaryl ring radical may be attached to the main structure at any heteroatom or carbon atom that results in the creation of a stable structure. Examples of such heteroaryl ring radicals include, but are not limited to, oxazolyl, imidazolyl, pyrrolyl, furanyl, triazinyl, pyridinyl, pyrimidinyl, pyrazinyl, benzofuranyl, indolyl, benzothiazolyl, benzoxazolyl, carbazolyl, quinazonyl and the like.

The term “heteroarylalkyl” unless otherwise specified refers to substituted or unsubstituted heteroaryl ring radical directly bonded to an alkyl group. The heteroarylalkyl radical may be attached to the main structure at any carbon atom in the alkyl group that results in the creation of a stable structure, wherein the heteroaryl and alkyl are the same as defined earlier.

The term “heterocyclylalkyl” unless otherwise specified refers to substituted or unsubstituted heterocyclic ring radical directly bonded to an alkyl group. The heterocyclylalkyl radical may be attached to the main structure at any carbon atom in the alkyl group that results in the creation of a stable structure wherein the heterocyclyl and alkyl are the same as defined earlier.

Unless otherwise specified, the term “substituted” as used herein refers to substitution with any one or any combination of the following substituents: hydroxy, halogen, carboxyl, cyano, nitro, oxo (═O), thio (═S), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkylalkyl, substituted or unsubstituted cycloalkenylalkyl, substituted or unsubstituted cycloalkenyl, substituted or unsubstituted amino, substituted or unsubstituted heteroaryl, substituted or unsubstituted heterocyclylalkyl ring, substituted or unsubstituted heteroarylalkyl, substituted or unsubstituted heterocyclic ring, substituted or unsubstituted guanidine, —COOR^{x}, —C(O)R^{x}, —C(S)R^{x}, —C(O)NR^{x}R^{y}, —C(O)ONR^{x}R^{y}, —NR^{x}CONR^{y}R^{z}, —N(R^{x})SOR^{y}, —N(R^{x})SO₂R^{y}, —(═N—N(R^{x})R^{y}), —NR^{x}C(O)OR^{y}, —NR^{x}R^{y}, —NR^{x}C(O)R^{y}, —NR^{x}(S)R^{y}, —NR^{x}C(S)NR^{y}R^{z}, —SONR^{x}R^{y}, —SO₂NR^{x}R^{y}, —OR^{x}, —OR^{x}C(O)NR^{y}R^{z}, —OR^{x}(O)OR^{y}, —OC(O) R^{x}, —OC(O)NR^{x}R^{y}, —R^{x}NR^{y}C(O)R^{z}, —R^{x}OR^{y}, —R^{x}C(O)OR^{y}, —R^{x}C(O)NR^{y}R^{z}, —R^{x}C(O)R^{y}, —R^{x}OC(O)R^{y}, —SR^{x}, —SOR^{x}, —SO₂R^{x}, and —ONO₂, wherein R^{x}, R^{y} and R^{z} are independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted heterocyclic ring, substituted or unsubstituted heterocyclylalkyl, or substituted or unsubstituted amino. According to one embodiment, the substituents in the aforementioned “substituted” groups cannot be further substituted. For example, when the substituent on “substituted alkyl” is “substituted aryl”, the substituent on “substituted aryl” cannot be “substituted alkenyl”.

Human SCD-1 has been described in detail, including full cDNA sequence and partial promoter sequences. For information, please see U.S. Pat. No. 6,987,001, which is incorporated herein by reference in its entirety and for the purpose of disclosing the SCD-1 particulars. SCD-1 expression has been observed in liver, eyelid, white adipose tissue and skin of wild type mice and liver, skin, adipose tissue and cornea of human. (for example, see Miyazaki et al., J Nutr. 2001 September; 131(9):2260-8; Zhang et al., Biochem. J. 1999 340, 255-264).

While the invention is not limited to any specific theory, the inventors of the present patent application unexpectedly discovered that certain SCD-1 compounds may exhibit selective activity in a target tissue in comparison with a reference tissue. The definition of the “target tissue” and “reference tissue” are set forth herein above. The inventors realized that substances having greater SCD-1 inhibitory activity in the target tissue than in the reference tissue shall be desirable for treating conditions associated with diseases related to the target tissue and/or associated therewith out significantly affecting the condition of the reference tissue. Again, while the invention is not limited to any specific theory, the inventors realized that such selective SCD-1 inhibitors would have desired activity profile for the disease process associated with the target tissue while having minimized/reduced side effects associated with activity in the reference tissue. For example, the inventors were aware that inhibition of SCD-1 enzyme in the liver is desirable with respect to SCD-1 inhibitor activity profile. The inventors were also aware that simultaneous inhibition of SCD-1 activity in skin and cornea may be undesirable because it could lead to side effects associated with the skin, such as alopecia (skin hair loss) and dry eye and corneal opacity. This led the inventors to search for substances that selectively inhibit SCD-1 enzyme in the liver while minimizing SCD-1 inhibition in the skin and cornea. While of lesser importance, the inventors also were led to search for substances that selectively inhibit SCD-1 enzyme in skin and/or cornea, while minimizing SCD-1 inhibition in liver and/or other non-target tissues.

Herein provided and described below an assay for identifying substances which exhibit such selective activity. Thus, there is provided a method of identifying liver-selective SCD-1 inhibitors, the method including:
(a) assaying a test compound for SCD-1 inhibition in whole liver cells,
(b) assaying the test compound for SCD-1 inhibition in whole skin cells, and/or whole corneal epithelial cells, and
(c) selecting the test compound as a selective inhibitor of liver SCD-1 if the ratio of the EC₃₅ of the test compound in whole skin cells and/or corneal cells to that in whole liver cells is at least 1.2:1.

Also provided is a method of identifying skin-selective SCD-1 inhibitors, the method including:
(a) assaying a test compound for SCD-1 inhibition in whole skin cells,
(b) assaying said test compound for SCD-1 inhibition in whole liver cells and/or in whole corneal epithelial cells, and
(c) selecting the test compound as a selective inhibitor skin SCD-1 inhibitor if the ratio of the EC₃₅ in whole liver cells and/or corneal cells to that in whole skin cells is at least 1.2:1.

Also provided is a method of identifying cornea-selective SCD-1 inhibitors, the method including:
(a) assaying a test compound for SCD-1 inhibition in whole corneal epithelial cells,
(b) assaying said test compound for SCD-1 inhibition in whole skin cells and/or in whole liver cells, and
(c) selecting the test compound as a selective corneal SCD-1 inhibitor if the ratio of the EC₃₅ in whole liver cells and/or skin cells to that in whole corneal epithelial cells is at least 1.2:1.

The steps a) and b) for the methods of identification described above involve determining SCD-1 inhibitory activity of the test substance, preferably, by measuring its EC₃₅ levels. The assay for determining the SCD-1 inhibitory activity may be any assay known in the art, for example, the assays set forth in U.S. Pat. No. 6,987,001, columns 12, 13, 14, 15, 16, 17, 18 and 19, which are incorporated by reference for the purpose stated. Alternatively, the screening assay may include the steps of:
(a) desaturating 9,10 ³H stearoyl CoA substrate by whole cells expressing SCD-1, in presence or absence of a test agent to form a fatty acyl CoA product and ³H water;
(b) removing background radioactivity from un-utilized substrate in the cell supernatant; and
(c) measuring radioactivity of the labeled water product as an indicator of SCD-1 enzymatic activity.

Background radioactivity from un-utilized substrate in the cell supernatant can be removed by several means known in the art. The applicants have found that the use of an ion exchange resin, more particularly an anion exchange resin, can efficiently remove the background radioactivity from the cell supernatant. One such resin is the Dowex® resin commercially available from Sigma-Aldrich (St. Louis, Mo.) and Dow Chemical (Midland, Mich.). Methods to measure radioactivity of the labeled water product are known in the art. Such methods include the use of scintillation counters and beta counters-Non-limiting examples of the assay for identifying selective SCD-1 inhibitors are described further at the end of the present specification.

Preferably, the methods described herein involve identifying substances with significantly increased inhibitory activity in the target tissue in comparison with reference tissue. The greater the difference in activity, the greater the desirability of the identified compound. Preferably, identified compounds have ratio of EC₃₅ in the reference tissue versus that in the target tissue greater than 2:1, more preferably, greater than 3:1, more preferably, greater than 4:1, yet more preferably, greater than 5:1.

While the invention is not limited by any specific theory, it is believed that compounds identified using the above-described methods have particular utility in achieving desired biological effects in a biological subject. Thus, there is provided a method of inhibiting lipid metabolism that proceeds via an SCD-1-mediated pathway in a subject by administering to the subject an inhibitory effective amount of a selective SCD-1 inhibitor that exhibits increased SCD-1 inhibitory activity in the target tissue in comparison with the SCD-1 activity in a reference tissue. Also provided is a method of decreasing serum levels of at least one lipid in a subject in need thereof via administering to the subject an inhibitory effective amount of a selective SCD-1 inhibitor that exhibits increased SCD-1 inhibitory activity in a target tissue in comparison with SCD-1 activity in a reference tissue. The preferred lipids are triglycerides, LDL, HDL, VLDL and cholesterol.

The subject may be any biological body, such as a cell or a mammal, including a human. As described herein above, the preferred target tissue is liver and the preferred reference tissue is skin or cornea. While the invention is not limited to any specific theory, liver-selective SCD-1 inhibitors contemplated herein are believe to function by decreasing conversion of saturated fatty acids to unsaturated fatty acids in liver cells to a greater extent than in skin cells and/or corneal cells. Particularly contemplated fatty acids are palmitoyl CoA and stearoyl CoA.

To practice the methods described herein above, the preferred SCD-1 inhibitors possess the ratio of EC₃₅ for the in skin cells to that in liver cells greater than 1, more preferably, at least 1.25:1, yet more preferably, at least 2:1, most preferably, at least 5:1.

The liver-selective SCD-1 inhibitors contemplated herein may be any biologically-active molecule, non-limiting examples of which include a protein, an antibody, or a small pharmaceutical molecule. Preferably, the selective SCD-1 inhibitor is a small pharmaceutical molecule in the form of a free compound or pharmaceutically acceptable salt thereof.

Particularly contemplated are compounds of the formula (I), which are incorporated herein as reference to our U.S. Provisional Application No. 61/082,048, filed on Jul. 18, 2008 and U.S. Provisional Application No. 61/087,833, filed on Aug. 12, 2008.

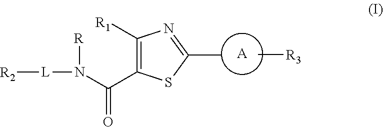

wherein,

A can be group selected from

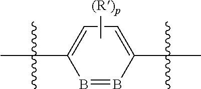

### or —C≡C—

at each occurrence B can be independently selected from C or N with proviso that at least one B is N;

R can be hydrogen, substituted or unsubstituted alkyl, substituted or unsubstituted cycloalkyl;

R′ can be is hydrogen, cyano, nitro, halogen, hydroxyl, substituted or unsubstituted alkyl, substituted or unsubstituted alkenyl, substituted or unsubstituted alkynyl;

‘p’ is an integer selected from 1-4;

R₁ can be hydrogen, substituted or unsubstituted alkyl, substituted or unsubstituted cycloalkyl, or haloalkyl;

R₂ can be substituted or unsubstituted aryl, substituted or unsubstituted heteroaryl substituted or unsubstituted cycloalkyl, substituted or unsubstituted heterocyclyl;

R₃ can be substituted or unsubstituted aryl, substituted or unsubstituted heteroaryl substituted or unsubstituted arylalkyl, substituted or unsubstituted heteroarylalkyl, substituted or unsubstituted cycloalkyl, substituted or unsubstituted heterocyclyl, substituted or unsubstituted cycloalkylalkyl, substituted or unsubstituted heterocyclylalkyl; and

L is alkylene linker which may be further substituted with halogen or alkyl.

Pharmaceutically acceptable salts of the compounds of the formula (I), are also contemplated. Likewise, pharmaceutically acceptable solvates, including hydrates, of the compounds of the formula (I) are contemplated. It should be understood that the structurally encompasses all stereoisomers, including enantiomers and diastereomers, that may be contemplated from the chemical structure of the genus described herein.

Preferred are compounds described in tables 2 to 4. To practice the methods described herein above, the selective SCD-1 inhibitors should be present in inhibitory effective amount, namely, the amount at which inhibition of SCD-1 enzyme can be measured via currently accepted measurement methodologies, such as for examples the assays described herein.

Selective SCD-1 inhibitors contemplated in the use of methods described herein may exhibit selective activity via several contemplated and considered mechanisms of action. While the invention is not limited by any specific theory, separately contemplated are SCD-1 inhibitors that inhibit SCD-1 gene expression, SCD-1 translation, or directly inhibit action of SCD-1 enzyme, or selectively exhibits permeability into target tissue.

Also provided is method of treating a disease or condition, the treatment of which is effected or facilitated by inhibiting SCD-1, in a subject in need of said treatment, the method including administering to the subject a therapeutically effective amount of a selective SCD-1 inhibitor that exhibits increased SCD-1 inhibitory activity in a target tissue in comparison with SCD-1 activity in a reference tissue.

Contemplated diseases and/or conditions include obesity or related conditions; diabetes (including Type I and Type II diabetes); diabetic complications; glucose tolerance; hyperinsulinemia; insulin sensitivity or resistance; metabolic syndromes; cardiovascular diseases including, for example, atherosclerosis, lipidemia, dyslipidemia, elevated blood pressure, microalbuminemia, hyperuricaemia, hypercholesterolemia, hyperlipidemias, hypertriglyceridemias, arteriosclerosis or combination thereof or any combination these diseases, disorders, conditions and/or syndromes thereof; the disease or condition related to serum levels of triglyceride, LDL, HDL, VLDL, and/or total cholesterol.

Obesity related syndromes, disorders and diseases include, but are not limited to, obesity as a result of (i) genetics, (ii) diet, (iii) food intake volume, (iv) a metabolic disorder, (v) a hypothalmic disorder, (vi) age, (vii) abnormal adipose mass distribution, (viii) abnormal adipose compartment distribution, (ix) compulsive eating disorders, and (x) motivational disorders which include the desire to consume sugars, carbohydrates, alcohols or drugs or any ingredient with hedonic value. Symptoms associated with obesity related syndromes, disorders, and diseases include, but are not limited to, reduced activity. Obesity also increases the likelihood of sleep apnea, gallstones, osteoporosis and certain cancers. (Agnieszka obrzyn and James M Ntambi 2004, Trends Cardiovasc Med, 14, 77-81; Jiang et al., 2005, Journal of clinical investigation, 115, 1030-1038).

Diabetes related syndromes, disorders and diseases include, but are not limited to, glucose dysregulation, insulin resistance, glucose intolerance, hyperinsulinemia, dyslipidemia, hypertension, obesity, and hyperglycemia. (James M Ntambi and Makoto Miyazaki 2003, Current Opinion in Lipidology, 14, 255-261).

Cardiovascular diseases include, but are not limited to, (i) coronary artery disease, (ii) atherosclerosis, (iii) heart disease, (iv) hypercholesterolemia, (v) hypertriglyceridemia, (vi) hypertriglyceridemia secondary to another disorder or disease (such as hyperlipoproteinemias), (vii) hyperlipidemia, (viii) disorders of serum levels of triglycerides, VLDL, HDL, and LDL, (ix) cholesterol disorders, (x) cerebrovascular disease (including but not limited to, stroke, ischemic stroke and transient ischemic attack (TIA)), (xi) peripheral vascular disease, and (xii) ischemic retinopathy. Metabolism related syndromes, disorders or diseases include, but are not limited to, (i) metabolic syndrome, (ii) dyslipidemia, (iii) elevated blood pressure, (iv) insulin sensitivity or resistance, (v) Type II diabetes, (vi) Type I diabetes, (vii) diabetic complications, (viii) increased abdominal girth, (ix) glucose tolerance, (x) microalbuminemia, (xi) hyperuricaemia, (xii) hyperinsulinemia, (xiii) hypercholesterolemia, (xiv) hyperlipidemias, (xv) atherosclerosis, (xvi) hypertriglyceridemias, (xvii) arteriosclerosis and other cardiovascular diseases, (xviii) osteoarthritis, (xix) dermatological diseases, (xx) sleep disorders (e.g., disturbances of circadian rhythm, dysomnia, insomnia, sleep apnea and narcolepsy), (xxi) cholelithiasis, (xxii) hepatomegaly, (xxiiii) steatosis, (xxiv) syndrome X, (xxv) abnormal alanine aminotransferase levels, (xxvi) polycystic ovarian disease, and (xxvii) inflammation. (Paul Cohen, James M Ntambi and Jaffrey M Friedman 2003, Current Drug targets-Immune, Endocrine and Metabolic Disorders, 3, 271-280)

Non-alcoholic fatty liver disease can manifest as hepatic steatosis (or fatty liver) and can progress to hepatitis, drug-induced hepatitis, hepatoma, fibrosis, hepatic cirrhosis, liver failure, non-alcoholic steatohepatitis, non-alcoholic hepatitis, acute fatty liver, and fatty liver of pregnancy.

Other disorders or diseases mediated by SCD include, but are not limited to, skin disorders, inflammation, pancreatitis, osteoarthritis, rheumatoid arthritis, cystic fibrosis, pre-menstrual syndrome, cancer, neoplasia, malignancy, metastases, tumours (benign or malignant), carcinogenesis, hepatomas, neurological diseases, psychiatric disorders, multiple sclerosis, and viral diseases and infections. Skin disorders include skin cancer, acne, atopic dermatitis, alopecia, hirsutism, and hypertrichosis.

In one embodiment, agents useful in practicing the invention, when administered to a patient in therapeutically effective doses, may increase HDL levels and/or decrease triglyceride levels and/or decrease LDL or non-HDL-cholesterol levels. In another embodiment, agents useful in practicing the invention, when administered to a patient in therapeutically effective doses, increase body lean mass and decrease obesity. In another embodiment, agents useful in practicing the invention, when administered to a patient in therapeutically effective doses, decrease hepatitic steatosis.

While the invention is not limited to any specific theory, the inventors of the present patent application unexpectedly discovered that certain SCD-1 compounds may exhibit selective activity in a target tissue in comparison with the reference tissue.

Particularly contemplated are compounds of the formula (I)

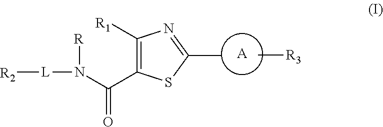

wherein,

A can be group selected from

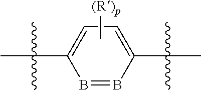

### or —C≡C—

at each occurrence B can be independently selected from C or N with proviso that atleast one B is N;

R can be hydrogen, substituted or unsubstituted alkyl, substituted or unsubstituted cycloalkyl;

R′ can be is hydrogen, cyano, nitro, halogen, hydroxyl, substituted or unsubstituted alkyl, substituted or unsubstituted alkenyl, substituted or unsubstituted alkynyl;

‘p’ is an integer selected from 1-4;

R₁ can be hydrogen, substituted or unsubstituted alkyl, substituted or unsubstituted cycloalkyl, or haloalkyl;

R₂ can be substituted or unsubstituted aryl, substituted or unsubstituted heteroaryl substituted or unsubstituted cycloalkyl, substituted or unsubstituted heterocyclyl;

R₃ can be substituted or unsubstituted aryl, substituted or unsubstituted heteroaryl substituted or unsubstituted arylalkyl, substituted or unsubstituted heteroarylalkyl, substituted or unsubstituted cycloalkyl, substituted or unsubstituted heterocyclyl, substituted or unsubstituted cycloalkylalkyl, substituted or unsubstituted heterocyclylalkyl;

L is alkylene linker which may be further substituted with halogen or alkyl.

Pharmaceutically acceptable salts of the compounds of the formula (I), are also contemplated. Likewise, pharmaceutically acceptable solvates, including hydrates, of the compounds of the formula (I) are contemplated. It should be understood that the structurally encompasses all stereoisomers, including enantiomers and diastereomers, that may be contemplated from the chemical structure of the genus described herein.

In one embodiment, the agents may be co-administered with other compounds to further enhance their activity, or to minimize potential side effects. As used in this application, co-administered refers to administering a selective SCD-1 modulating agent with a second medicinal, typically having a differing mechanism of action, using a dosing regimen that promotes the desired result. This can refer to simultaneous dosing, dosing at different times during a single day, or even dosing on different days. The compounds can be administered separately or can be combined into a single formulation. Techniques for preparing such formulations are described below.

The route of administration may be any route which effectively transports the active compound of the invention to the appropriate or desired site of action. Suitable routes of administration include, but are not limited to, oral, nasal, pulmonary, buccal, subdermal, intradermal, transdermal, parenteral, rectal, depot, subcutaneous, intravenous, intraurethral, intramuscular, intranasal, ophthalmic (such as with an ophthalmic solution) or topical (such as with a topical ointment). The oral route is preferred.

In order to exhibit the therapeutic properties described above, the agents need to be administered in a quantity sufficient to modulate SCD-1 biological activity in the desired tissue, without significantly modulating the SCD activity in an undesired tissue such that undesired side effects are not produced in the undesired tissue. In general, a dose of about 0.01 to 1000 mg/kg/day can be administered in single or multiple doses. This amount can vary depending upon the particular disease/condition being treated, the severity of the patient's disease/condition, the patient, the particular compound being administered, the route of administration, and the presence of other underlying disease states within the patient, etc.

The pharmaceutical composition useful to practice the present invention comprises one or more agents having the aforementioned SCD tissue selectivity and one or more pharmaceutically acceptable excipients, carriers, diluents or a mixture thereof. The agents may be identified by the screening assay of the present invention. The SCD tissue selective agents may be associated with one or more pharmaceutically acceptable excipients, carriers, diluents or mixture thereof in the form of a capsule, sachet, paper or other container.

Examples of suitable carriers include, but are not limited to, water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethyl cellulose and polyvinylpyrrolidone.

The carrier or diluent may include a sustained release material, such as glyceryl monostearate or glyceryl distearate, alone or mixed with a wax.

The pharmaceutical composition may also include one or more pharmaceutically acceptable auxiliary agents, wetting agents, emulsifying agents, suspending agents, preserving agents, salts for influencing oxmetic pressure, buffers, sweetening agents, flavoring agents, colorants, or any combination of the foregoing. The pharmaceutical composition of the invention may be formulated so as to provide quick, sustained, or delayed release of the active ingredient after administration to the subject by employing methods known in the art.

The pharmaceutical compositions may be prepared by conventional techniques, e.g., as described in Remington: The Science and Practice of Pharmacy, 20^{th} Ed., 2003 (Lippincott Williams & Wilkins). For example, the active compound is mixed with a carrier, or diluted by a carrier, or enclosed within a carrier, which may be in the form of an ampoule, capsule, sachet, paper, or other container. When the carrier serves as a diluent, it may be a solid, semi-solid, or liquid material that acts as a vehicle, excipient, or medium for the active compound. The active compound is adsorbed on a granular solid container, for example, in a sachet.

The pharmaceutical compositions may be in conventional forms, for example, capsules, tablets, aerosols, solutions, suspensions or products for topical application.

Solid oral formulations include, but are not limited to, tablets, capsules (soft or hard gelatin), dragees (containing the active ingredient in powder or pellet form), troches and lozenges. Tablets, dragees, or capsules having talc and/or a carbohydrate carrier or binder or the like are particularly suitable for oral application. Preferable carriers for tablets, dragees, or capsules include lactose, cornstarch, and/or potato starch. A syrup or elixir is used in cases where a sweetened vehicle is employed.

Liquid formulations include, but are not limited to, syrups, emulsions, soft gelatin and sterile injectable liquids, such as aqueous or non-aqueous liquid suspensions or solutions.

For parenteral application, particularly suitable are injectable solutions or suspensions, preferably aqueous solutions with the active compound dissolved in polyhydroxylated castor oil.

Suitable doses of the compounds for use in treating the diseases and disorders described herein can be determined by those skilled in the relevant art. Therapeutic doses are generally identified through a dose ranging study in humans based on preliminary evidence derived from the animal studies. Doses must be sufficient to result in a desired therapeutic benefit without causing unwanted side effects. Mode of administration, dosage forms, suitable pharmaceutical excipients, diluents or carriers can also be well used and adjusted by those skilled in the art. All changes and modifications are envisioned within the scope of the present invention.

### EXAMPLES

### Example 1

### Whole Cell Based Assay to Screen for Agents that Modulate SCD-1 Activity

An assay was developed using whole cells to screen for agents that modulate SCD-1 activity. Three cell lines namely human liver cell line (hepatocellular carcinoma HepG2 ATCC HB 8065), human skin cell line (epidermal carcinoma A431 ATCC CRL 1555) and human corneal epithelial progenitor (HCEP) cells (CELLNTEC advanced cell systems) were used. HepG2 cells were seeded in a 24 well plate in complete MEM medium (Hyclone). A431 cells were seeded in a 12 well plate in complete DMEM medium (Sigma). HCEP cells were seeded in a 96 well plate in defined corneal epithelial medium CnT 20 (CELLNTEC advanced cell systems). HepG2 cells were induced with LXR agonist T0901317 (Cayman chemicals) in medium containing high glucose and fatty acid free BSA for 3 days with media change every day (Wang et al, (2004) Journal of Lipid Research, 45, 972-980). A431 cells were not induced. (3). The cells were preincubated in plain medium with the known SCD-1 modulators namely CLA (conjugated linoleic acid) (Sigma) and TSA (9 thiastearic acid) (Cayman chemicals) for 15-30 minutes at 37 deg C. and further challenged with 0.25 to 7.5 μCi tritiated Stearoyl CoA (American Radiolabelled chemicals). The cells were incubated for last 6 hours in 5% CO₂ incubator at 37 deg C. or in specific embodiments for 4 hours to 30 hours. At the end of the incubation, the incubation medium was collected into tubes. Dowex AG 1-X8 resin (Biorad laboratories) pre-equilibrated with distilled ethanol:water was added to the tubes to separate the unconsumed substrate from the labelled water. The tubes were then vortexed for 5 minutes and centrifuged at 14000 rpm for 20 minutes at room temperature. The supernatant from the tubes was mixed with the scintillation fluid and radioactivity counted in the Packard topcount scintillation counter (Perkin Elmer). Counts obtained were normalised per million cells. Inhibition of enzyme activity was calculated as the percent of maximum reaction control that contained no test compound. EC₃₅ value and percentage inhibition of SCD-1 by CLA and TSA using this cell based assay is given in Table 1.

TABLE 1
Inhibition of SCD-1 activity by CLA and
TSA in whole cell based assay (EC₃₅)

| | HepG2 | A431 | HCEP |
|---|---|---|---|
| CLA | 22.66 μM | 16.35 μM | ~80% at 300 μM |
| | (~80.01% at 300 μM) | (~91% at 300 μM) | |
| TSA | ~100 μM | ~100 μM | Not available |
| | (52% at 100 μM) | (58% at 100 μM) | |

From Table 1, it can be seen that CLA showed an EC₃₅ of 22.66 μM and 16.35 μM in liver cells and skin cells respectively. TSA showed 52% inhibition of SCD-1 in skin cells and 58% inhibition in liver cells at 100 μM. The inhibition of cellular SCD-1 activity produced by CLA in HepG2 cells is in agreement with the one reported in literature using MCF-7 and MDA-MB-231 cells and TLC based method (Choi et al., Biochem Biophys Res Commun. 2002 Jun. 21; 294(4):785-90) and thus validates the whole cell assay method developed by us.

### Example 2

### Whole Cell Based Assay to Screen for Agents that Differentially Modulate SCD-1 Activity Present in Different Tissues

The whole cell based assay was adapted to screen for agents that differentially modulate SCD-1 activity present in different tissues. Three cell lines namely human liver cell line (hepatocellular carcinoma HepG2), human skin cell line (epidermal carcinoma A431) and human corneal epithelial progenitor (HCEP) cells were used. HepG2 cells were seeded in a 24 well plate in complete MEM medium. A431 cells were seeded in a 12 well plate in complete DMEM medium. HCEP cells were seeded in a 96 well plate in defined corneal epithelial medium CnT 20. HepG2 cells were induced with LXR agonist T0901317 in medium containing 4.5 gm/litre glucose and 0.1% fatty acid free BSA for 3 days with media change every day (Wang et al, (2004) Journal of Lipid Research, 45, 972-980). A431 cells and HCEP cells were not induced. (3). The cells were preincubated in plain medium 0.1 nM to 10 μM concentrations potential SCD-1 modulator compounds for 15-30 minutes at 37 deg C. and further challenged with 0.25 to 7.5 μCi tritiated Stearoyl CoA. The cells were incubated for last 6 hours in 5% CO₂ incubator at 37 deg C. or in specific embodiments for 4 hours to 30 hours. At the end of the incubation, the incubation medium was collected into tubes. Dowex AG 1-X8 resin pre-equilibrated with distilled ethanol:water was added to the tubes to separate the unconsumed substrate from the labelled water. The tubes were then vortexed for 5 minutes and centrifuged at 14000 rpm for 20 minutes at room temperature. The supernatant from the tubes was mixed with the scintillation fluid and radioactivity counted in the Packard topcount scintillation counter. Counts obtained were normalised per million cells. Inhibition of enzyme activity was calculated as the percent of maximum reaction control that contained no test compound. For active compounds, EC₃₅ was calculated from dose response curve by non linear regression analysis using GraphPadPRISM software. The ratio of EC₃₅ for skin cells to EC₃₅ for liver cells was calculated. The EC₃₅ values for liver, skin and corneal cells along with their ratios are given in Tables 2-5.

TABLE 2
Compounds Selective For Liver Over Skin

| | | IC₅₀ | hLiver | hSkin | Fold |
|---|---|---|---|---|---|
| SN | Structure | (nM) | EC₃₅ (nM) | EC₃₅ (nM) | Skin:liver |
| 1 | 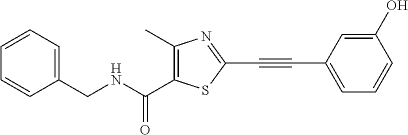 | <1000 | 3440 | 6900 | 2.00 |
| 2 | 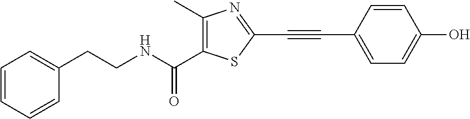 | <500 | 490 | 982 | 2.00 |
| 3 | 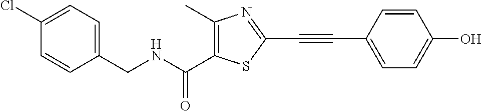 | <100 | 698 | >10,000 | >15 |

TABLE 3
Compounds Selective For Skin Over Liver

| | | IC₅₀ | hLiver | hSkin | Fold |
|---|---|---|---|---|---|
| SN | Structure | (nM) | EC₃₅ (nM) | EC₃₅ (nM) | Liver:skin |
| 1 | 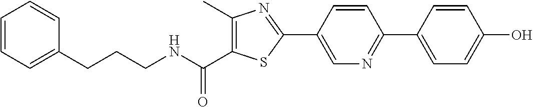 | <100 | 266 | 179.9 | 1.47 |
| 2 | 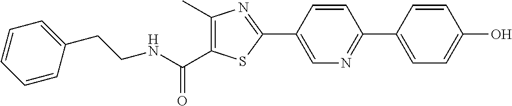 | <100 | 437.5 | 167.7 | 2.60 |

TABLE 4
Compounds Selective For Cornea Over Liver

| | | IC₅₀ | hLiver | hCornea | Fold |
|---|---|---|---|---|---|
| SN | Structure | (nM) | EC₃₅ (nM) | EC₃₅ (nM) | Liver:Cornea |
| 1 | 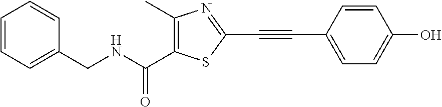 | <250 | 363 | 218 | 1.66 |
| 2 | 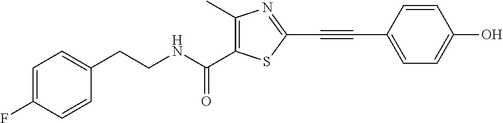 | <100 | 480.20 | 53.23 | 9.02 |

TABLE 5
Non Selective Compounds

| | | IC₅₀ | hLiver | hSkin | FOLD |
|---|---|---|---|---|---|
| SN | Structure | (nM) | EC₃₅ (nM) | EC₃₅ (nM) | skin:liver |
| 1 | 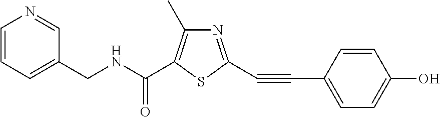 | <1000 | 2300 | 2140 | 0.93 |
| 2 | 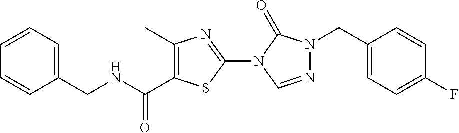 | <100 | 39.5 | 37.8 | 0.95 |
| 3 | 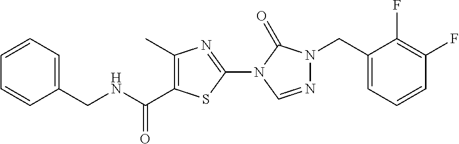 | <100 | 29.43 | 33.83 | 1.15 |

All patents and other publications cited in this application are hereby incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention. The following examples and biological data are being presented in order to further illustrate the invention. This disclosure should not be construed as limiting the invention in any manner.

We claim:

1. A method of inhibiting lipid metabolism that proceeds via a Stearoyl-CoA Desaturase-1 (SCD-1) mediated pathway in a subject, said method comprising administering to said subject an inhibitory effective amount of a selective SCD-1 inhibitor that exhibits increased SCD-1 inhibitory activity in a target tissue in comparison with SCD-1 activity in a reference tissue.

2. A method of decreasing serum levels of at least one lipid in a subject in need thereof, said method comprising administering to said subject an inhibitory effective amount of a selective SCD-1 inhibitor that exhibits increased SCD-1 inhibitory activity in a target tissue in comparison with SCD-1 activity in a reference tissue.

3. The method of claim 2, wherein said target tissue is selected from liver, skin and cornea.

4. The method of claim 2, wherein said reference tissue is selected from liver, skin and cornea.

5. The method of claim 2, wherein said target tissue is liver and said reference tissue is skin.

6. The method of claim 2, wherein said target tissue is liver and said reference tissue is cornea.

7. The method of claim 2, wherein said selective SCD-1 inhibitor decreases conversion of saturated fatty acids to unsaturated fatty acids in liver cells to a greater extent than in skin cells.

8. The method of claim 2, wherein said subject is a cell.

9. The method of claim 2, wherein said subject is a mammal, which includes human.

10. (canceled)

11. The method of claim 7, wherein said fatty acid is selected from palmitoyl CoA and stearoyl CoA.

12. The method of claim 2, wherein said selective SCD-1 inhibitor is a small pharmaceutical molecule in the form of a free compound or pharmaceutically acceptable salt thereof.

13. (canceled)

14. The method of claim 5, wherein the ratio of EC₃₅ for the SCD-1 inhibitor in skin cells to that in liver cells is greater than 1.

15. The method of claim 14, wherein said ratio is at least 1.25:1.

16. The method of claim 14, wherein said ratio is at least 2:1.

17. (canceled)

18. The method of claim 2, wherein lipid is selected from the group consisting of triglycerides, LDL, HDL, VLDL and cholesterol.

19.-23. (canceled)

24. A method of treating a disease or condition selected from obesity, diabetes, glucose tolerance; hyperinsulinemia; insulin insensitivity or resistance, metabolic syndromes, and cardiovascular disease in a subject in need of said treatment, said method comprising administering to said subject a therapeutically effective amount of a selective SCD-1 inhibitor that exhibits increased SCD-1 inhibitory activity in a target tissue in comparison with SCD-1 activity in a reference tissue.

25.-32. (canceled)

33. The method of claim 24, wherein said cardiovascular disease is atherosclerosis, hypertension, lipidemia, dyslipidemia, elevated blood pressure, microalbuminemia, hyperuricaemia, hypercholesterolemia, hyperlipidemias, hypertriglyceridemias, or arteriosclerosis.

34. A method of identifying a liver-selective SCD-1 inhibitor, said method comprising:
(a) assaying a test compound for SCD-1 inhibition in whole liver cells,
(b) assaying said test compound for SCD-1 inhibition in whole skin cells and/or in whole corneal cells, and
(c) selecting the test compound as a selective inhibitor of liver SCD-1 if the ratio of the EC₃₅ of the test compound in whole skin cells and/or corneal cells to that in whole liver cells is at least 1.2:1.

35. The method of claim 34, wherein said ratio is at least 2:1.

36. The method of claim 34, wherein said ratio is at least 3:1.

37.-38. (canceled)

39. A method of identifying a skin-selective SCD-1 inhibitor, comprising:
(a) assaying a test compound for SCD-1 inhibition in whole skin cells,
(b) assaying said test compound for SCD-1 inhibition in whole liver cells, and/or in whole corneal cells,
(c) selecting the test compound as a selective inhibitor skin SCD-1 inhibitor if the ratio of the EC₃₅ in whole liver cells and/or corneal cells to that in whole skin cells is at least 1.2:1.

40. The method of claim 39, wherein said ratio is at least 2:1.

41. The method of claim 39, wherein said ratio is at least 3:1.

42.-43. (canceled)

44. A method of identifying a corneal-selective SCD-1 inhibitor, comprising:
(a) assaying a test compound for SCD-1 inhibition in whole corneal cells,
(b) assaying said test compound for SCD-1 inhibition in whole liver cells, and/or in whole skin cells,
(c) selecting the test compound as a selective corneal SCD-1 inhibitor if the ratio of the EC₃₅ in whole liver cells and/or skin cells to that in whole corneal cells is at least 1.2:1.

45. The method of claim 44, wherein said ratio is at least 2:1.

46.-57. (canceled)
